(19) United States
(12) Patent Application Publication
(10) Pub. No.: US 2008/0275082 A1
(43) Pub. Date: Nov. 6, 2008

(54) Pharmaceutical Composition

(76) Inventors: Jeffrey Brum, Collegeville, PA (US); Sisay Gebrekidan, King of Prussia, PA (US); Rennan Pan, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/088,647

(22) PCT Filed: Sep. 28, 2006
(86) PCT No.: PCT/US06/38067

§ 371 Date: Mar. 28, 2008

Related U.S. Application Data
(60) Provisional application No. 60/722,761, filed on Sep. 30, 2005.

(51) Int. Cl. A61K 31/47; A61P 25/28; A61P 9/00; A61P 11/00

(52) U.S. Cl. 514/312

(57) ABSTRACT

A novel pharmaceutical composition comprising the NK₃ receptor antagonist talnetant, povidone, erythritol and a surfactant, and a process for its preparation are disclosed. The use of erythritol as soluble filler in the preparation of a stable pharmaceutical composition is disclosed.

The present invention relates to the use of erythritol as a stable soluble filler in spray-dried pharmaceutical compositions and, in particular, to novel compositions comprising eythritol as excipient and the NK₃ receptor antagonist talnetant.

Talnetant, (S)-(−)-N-(α-ethylbenzyl)-3-hydroxy-2-phenylquinoline-4-carboxamide, (alternatively 3-hydroxy-2-phenyl-N-[(1S)-1-phenylpropyl]-4-quinolinecarboxamide), has the chemical structure (A).

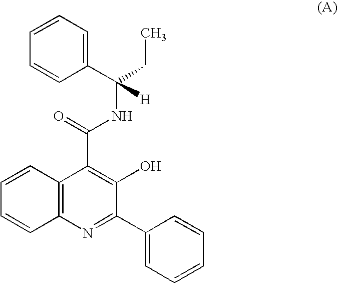

Talnetant, its preparation and its use in the treatment of pulmonary disorders, disorders of the central nervous system and neurodegenerative disorders are disclosed in published International Patent application WO 95/32948. Published International Patent applications WO 97/19927, WO 97/19928, WO 99/14196 and WO 02/094187 disclose additional therapeutic utilities for talnetant, pharmaceutically acceptable salts and processes for its preparation. WO 05/97077 discloses spray-dried compositions containing talnetant which have enhanced bioavailability. The above-mentioned patent applications are herein incorporated by reference as if each individual publication were specifically and individually indicated to be incorporated by reference herein as though fully set forth.

Talnetant has low aqueous solubility (approximately 0.03 mg/ml at pH 1 and 0.001 mg/ml, at pH 7.0). Typically drugs with low aqueous solubility are absorbed slowly across the walls of the gastrointestinal tract (GIT) due to poor dissolution of the solid in the GIT leading to a small diffusive driving force.

There are a number of different methods employed to improve absorption of a particular drug substance. It may be possible to develop so-called prodrugs or salts of the active agent, i.e. more soluble derivatives, by attaching a solubilizing group (e.g. phosphate, succinate or polyethylene glycol) to the drug, thereby taking advantage of the high solubility and dissolution rate of the derivative prodrug/salt. Alternatively, it is known to use physical formulation methods, such as use of amorphous drug or dispersion in a soluble carrier to increase the dissolution rate of the drug product and hence the absorption rate (J. H. Fincher, J. Pharm. Sci., 1968, 57, 1825 and G. L. Amidon et al., J. Pharm. Sci., 1980, 12, 1363).

A further alternative is to decrease the particle size of the drug. Decreasing the particle size increases the surface area of the drug particle, thereby increasing its dissolution rate.

A variety of processes have been developed to prepare fine particles of drug substance. Typically, dry milling techniques are used for the preparation of particulate medicaments (see E. L. Parrott, J. Pharm. Sci., 1974, 63, 813). Air jet milling and fluid energy milling (micronising) have been favoured because of the reduced risk of introducing contamination from mill materials. More recently, particles having a size of less than 1 μm have been obtained using wet milling processes. For example, published European Patent application EP-A-0 262 560 describes the use of wet milling techniques to prepare compositions containing benzoyl urea derivatives in which the average particle size is 1 μm or less. Provision of the fine particles is said to improve the absorbability from the GIT of the poorly water soluble benzoyl urea compounds thereby increasing their bioavailability. European Patent application EP-0 499 299 B1 describes a wet milling procedure for preparing particles of a crystalline drug substance, which particles have a surface modifier adsorbed on the surface in an amount sufficient to maintain an ‘effective average particle size’ of less than about 400 nm.

Aqueous dispersions obtained from wet milling processes may be used directly as a therapeutic agent if prepared under conditions of appropriate hygiene, for example, by using water and other components which meet Ph Eur/USP standards. For the preparation of formulations for use in human therapy, it is preferred that the aqueous dispersion is converted to a dry powder. This is suitably carried out by spray drying the resulting aqueous dispersion, typically collecting the product from the dryer using a cyclone separator. The resulting aqueous dispersion may also be spray granulated.

The objective of spray drying is to remove water from dispersions of drug particles so that the powder can be processed further to prepare capsule or tablet or other suitable oral dosage form. However, it is desirable that particles obtained from the spray dried powder are substantially the same size when dispersed in aqueous medium as the freshly-milled particles. If particles of the same size as the freshly-milled particles are obtained, it is referred to in the art (and hereinafter) as complete “recovery of particle size”.

On the other hand, it is also desirable that the form of excipients in the spray dried powder exists as a thermodynamically stable form so that the spray dried powder has a good storage stability. Typically mannitol or lactose have been previously used as a soluble filler in spray-dried pharmaceutical compositions. However, mannitol suffers from the disadvantage that it can exist in three different crystalline polymorphic forms: α, β and δ (see for example Burger, A., Henck, J. O., Hetz, S., Rollinger, J., Weissnicht, A., Stottner, H. J. Pharm Sci, 89, 457, (2000). Frequently, α-mannitol or δ-mannitol, or a mixture of α-mannitol and δ-mannitol forms, result from the spray drying process. These are metastable forms relative to β-mannitol which is the thermodynamically stable form at ambient conditions. Interconversion of mannitol forms is possible on storage of spray dried materials, or pharmaceutical dosage forms made from the spray dried materials. Lactose suffers from the disadvantage that it exists as an amorphous form in the spray dried materials, which may convert to a crystalline form on storage of spray dried materials, or pharmaceutical dosage forms made from the spray dried materials.

In addition, spray drying certain wet-milled dispersions of talnetant results in a poor recovery of particle size, i.e. a significant increase in particle size is seen when the spray dried materials are redispersed in aqueous media. Spray drying wet-milled dispersions of talnetant containing certain excipients may address this problem, and result in increased recovery of particle size and bioavailability. However, this may not necessarily address the stability issue, especially the form conversion of the soluble filler on storage, which frequently results in decreased particle size recovery.

For the commercial manufacture of many pharmaceutical actives and, in particular, for the manufacture of talnetant, storage stability, leading to reliable particle size recovery, is important. It has now been found that spray-dried pharmaceutical compositions, and in particular, a talnetant formulation composition containing crystalline erythritol addresses this issue, resulting in a stable crystalline form of soluble filler in the spray dried powder.

Thus, in a first aspect, the present invention provides the use of substantially crystalline erythritol as a stable soluble filler in a spray-drying process.

In another aspect, the present invention provides the use of substantially crystalline erythritol as a stable soluble filler in the preparation of spray-dried pharmaceutical compositions.

Unless otherwise stated, the word “stable” is intended to mean that the form of erythritol does not change in the spray dried materials from its input form.

In another embodiment, the present invention provides a spray-dried pharmaceutical composition comprising (i) a pharmaceutical active, (ii) povidone, (ii) substantially crystalline erythritol and (iv) a surfactant.

In a further embodiment, the present invention provides a pharmaceutical composition comprising (i) talnetant, (ii) povidone, (iii) substantially crystalline erythritol and (iv) a surfactant.

In one embodiment, talnetant is in the form of talnetant particles having a Dv90 in the range from 0.1 to 2.0 μm. As used herein, the term Dv90 refers to the value in micrometers at the 90^{th} percentile of a volume distribution derived from low angle laser light scattering (Malvern Mastersizer 2000). Similarly the Dv50 and Dv10 parameters refer to the 50^{th} and 10^{th} percentiles respectively of the same distribution.

To obtain talnetant particles having a Dv90 in the range from 0.1 to 2.0 μm, talnetant may be first wet-milled in any suitable aqueous, non-aqueous or organic solvent (e.g. an oil), and then spray dried. Suitable milling apparatus include conventional wet bead mills such as those manufactured by Nylacast (available from Nylacast Components, 200 Hastings Road, Leicester, LE5 0HL, UK), Netzsch (available from Erich NETZSCH GmbH & Co. Holding KG Gebrüder-Netzsch-Straβe 19, D-95100 Selb, Germany), Drais (available from Draiswerke, Inc, 40 Whitney Road, Mahwah, N.J. 07430, USA) and others. The milling chamber of the milling apparatus may be lined with or constructed from an abrasion-resistant polymer material. The milling chamber of the milling apparatus may be lined with or constructed from nylon. An example of a suitable milling chamber is described in International Patent Publication WO 02/00196. Suitable grinding media include glass beads and ceramic beads, for example, those made from rare earth oxide materials. The diameter of said grinding media is for example within the range 0.1 mm to 3 mm, suitably within the range 0.3 mm to 0.8 mm. The density of said grinding media is for example greater than 3 gcm⁻³, suitably within the range 5 to 10 gcm⁻³. Suitable spray drying and spray granulating techniques will be apparent to those skilled in the art (see for example, Gilbert S. Banker, “Modern Pharmaceutics, Drugs and the Pharmaceutical Sciences”, 1996 and references cited therein) and may be effected using a spray dryer, such as the Niro SD 6.3R Spray Dryer (Niro A/S, Gladsaxevej 305, 2860 Soeborg, Denmark), the Niro Mobile Minor, the Yamato GA-32 Spray Dryer (2-1-6 Nihonbashi Honcho, Chuo-ku, Tokyo, 103-8432, Japan) or a fluid bed granulator, such as the Glatt fluid bed granulator. Talnetant particles may be sized using conventional techniques known in the art, such as laser light diffraction and photon correlation spectroscopy.

Povidone (also known as polyvinyl pyrolidone or PVP) is an anti-agglomeration agent. Examples include Kollidon 30 and Plasdone K29/32.

Erythritol is a non cariogenic excipient used in a variety of pharmaceutical preparations, including in solid dosage form as a tablet filler (Bi Y X, Sunada Y, Yonezawa Y, Danjo K, Evaluation of rapidly disintegrating tablets prepared by a direct compression method. Drug Dev Ind Pharm 1999; 25(5); 571-581), and in coatings (Ohmori S, Ohno Y, Makino T, Kashihara T. Characteristics of erythritol and formulation of a novel coating with erythritol termed thin-layer sugarless coating. Int J Pharm 2004; 278(2); 447-457). It may also be used as a diluent in wet granulation in combination with moisture-sensitive drugs (Michaud J, Haest g. Erythritol: a new multipurpose excipient. Pharmaceut Technol Eur 2003; 15(10); 69-72).

Erythritol is a soluble carbohydrate which acts as a soluble carrier in the present invention. Examples include Eridex. The erythritol used in the present invention is substantially in the crystalline form, as reported in C. Ceccarelli, G. A. Jeffrey, R. K. McMullen, Acta Crystallogr B. Vol 36, p 3079 (1980). A particular benefit of the composition of the present invention is that the crystalline form of erythritol matches that of the input material prior to dissolution in the suspension and subsequent spray drying. Further, the erythritol is physically stable in this formulation, which contributes to storage stability and stable particle size recovery.

The erythritol may be dissolved in the dispersion prior to wet milling. Alternatively, it may be dissolved in the wet milled dispersion before spray drying.

The surfactant in the composition of the present invention may be an ionic surfactant or a non-ionic surfactant. If an ionic surfactant is used, it may be an anionic surfactant or a cationic surfactant. Examples of anionic surfactants include alkyl sulfates such as sodium lauryl sulfate, and dioctyl sodium sulfosuccinate (docusate sodium). Examples of cationic surfactants include cetyl pyridinium chloride and cetyl trimethylammonium bromide. In an embodiment, the surfactant is an anionic surfactant. In a further embodiment, the surfactant is sodium lauryl sulfate or dioctyl sodium sulfosuccinate (docusate sodium). In a still further embodiment the surfactant is sodium lauryl sulfate.

In one embodiment, the surfactant is a non-ionic surfactant. Examples of non-ionic surfactants include POE alkylphenols, POE straight-chain alcohols, POE polyoxypropylene glycols, POE mercaptans, long-chain carboxylic acid esters such as glyceryl and polyglyceryl esters of natural fatty acids, propylene glycol, sorbitol and POE sorbitol esters, polyoxyethylene glycol esters etc. In a further embodiment, the non-ionic surfactant is a POE polyoxypropylene glycol.

In one embodiment, the concentration of surfactant in the spray dried composition is 0.5 to 50.0% by weight of talnetant. In an embodiment, the concentration of surfactant in the dispersion prior to spray drying is 0.05 to 10.0% by weight of dispersion. In a further embodiment, the concentration of surfactant in the dispersion prior to spray drying is 0.05 to 2.0%.

In one embodiment, the dispersion contains 0.001 to 0.1 moles of ionic surfactant per mole of talnetant. In a further embodiment, the dispersion contains 0.005 to 0.05 moles of surfactant per mole of talnetant.

The composition of the present invention may contain further suitable pharmaceutically acceptable excipients, which may be added. Suitable excipients are described in the Handbook of Pharmaceutical Excipients, Pharmaceutical Press, 5^{th} Edition, 2006, published by The American Pharmaceutical Association and The Royal Pharmaceutical Society of Great Britain. Examples of further excipients include stablilisers to maintain the particles in suspension.

The composition of the present invention may be subjected to dry milling, wet milling and/or spray drying. As discussed above, for wet milling, typically water is used as an aqueous medium, and then removed by spray drying to obtain a spray dried powder. Thus in one embodiment, the composition of the present invention also comprises water. In one embodiment, the composition of the present invention comprises 50% to 75% water by unit formula % w/w.

In one embodiment, the composition of the present invention consists of:

| Ingredients | Unit Formula % w/w |
|---|---|
| Talnetant | 20.0 |
| Sodium Lauryl Sulfate | 0.3 |
| Povidone | 3.0 |
| Erythritol | 7.5 |
| Water | 69.2 |

In one embodiment, the composition of the present invention consists of:

| Ingredients | Unit formula % w/w |
|---|---|
| Talnetant | 20.0 |
| Sodium Lauryl Sulfate | 0.3 |
| Povidone | 1.7 |
| Erythritol | 10.0 |
| Water | 68.0 |

In one aspect, there is provided a process for the preparation of a spray-dried composition, the process comprising:

(1) wet milling a dispersion of (i) pharmaceutical active, (ii) povidone, (iii) substantially crystalline erythritol and (iv) a surfactant, and then
(2) spray drying or spray granulating the resulting dispersion.

In a further aspect, there is provided a process for the preparation of a spray-dried composition, the process comprising:

(1) wet milling a dispersion of (i) talnetant, (ii) povidone, (iii) substantially crystalline erythritol and (iv) a surfactant, and then
(2) spray drying or spray granulating the resulting dispersion.

In another aspect, the present invention provides a spray dried composition obtainable according to the process as defined above. Thus the present invention provides a spray dried composition comprising (i) a pharmaceutical active, (ii) povidone, (iii) substantially crystalline erythritol and (iv) a surfactant.

In another aspect, the present invention provides a spray dried composition obtainable according to the process as defined above. Thus the present invention provides a spray dried composition comprising (i) talnetant, (ii) povidone, (iii) substantially crystalline erythritol and (iv) a surfactant.

All features and embodiments of the first aspect of the present invention apply to the other aspects mutatis mutandis.

The composition of the present invention may be administered to the subjects without further processing. However it will generally be formulated into other dosage forms in conjunction with further pharmaceutically acceptable excipients selected with regard to the desired dosage form. These further excipients will typically be added to the spray dried composition after spray drying.

Thus in one embodiment, there is provided a dosage form comprising a composition defined in the first aspect.

In one embodiment, the dosage form is administered orally. Oral administration will typically involve swallowing so that the compound enters the GIT. Dosage forms for oral administration include solid formulations such as tablets, capsules (containing particulates, powders or non-aqueous suspension), sachets, vials, powders, granules, lozenges, reconstitutable powders and liquid preparations (such as suspensions, emulsions and elixirs).

Oral dosage forms may contain further excipients such as binding agents (for example syrup, acacia, gelatin, sorbitol, starch, PVP, HPMC, and tragacanth); fillers (for example lactose, sugar, maize-starch, calcium phosphate, sorbitol and glycine); tabletting lubricants (for example magnesium stearate); glidants (for example colloidal silicon dioxide such as Cab-O-Sil M-5P) and disintegrants [for example starch, crospovidone (Polyplasdone XL), croscarmellose sodium, sodium starch glycollate and microcrystalline cellulose (Avicel PH 102)]. In addition, the oral dosage form may contain preservatives, anti-oxidant, flavours, granulation binders, wetting agents and colorants.

In one embodiment, the dosage form for oral administration is a capsule. Excipients suitable for preparing liquid dosage forms include: suspending agents (for example sorbitol, syrup, methyl cellulose, gelatin, hydroxyethylcellulose, carboxymethylcellulose, aluminium stearate gel and hydrogenated edible fats); emulsifying agents (for example lecithin, sorbitan monooleate and acacia); aqueous or non-aqueous vehicles, which include edible oils (for example almond oil and fractionated coconut oil), oily esters (for example esters of glycerine and propylene glycol), ethyl alcohol, glycerine, water and normal saline; preservatives (for example methyl, propyl p-hydroxybenzoate and sorbic acid); and if desired conventional flavouring or colouring agents.

In another embodiment, the dosage form for oral administration is a tablet. Tablets may be prepared using standard technology familiar to the formulation scientist, for example by direct compression, granulation, melt congealing and extrusion. The tablet may be coated or uncoated. The tablet may be formulated to be immediate or controlled release. Controlled release formulations include delayed-, sustained-, pulsed- or dual-release. Suitable tabletting excipients are described in the Handbook of Pharmaceutical Excipients, Pharmaceutical Press, 5^{th} Edition 2006, published by The American Pharmaceutical Association and The Royal Pharmaceutical Society of Great Britain. Typical tabletting excipients include: carriers [for example microcrystalline cellulose (Avicel PH 102)], lubricating agents (for example magnesium stearate), binding agents, wetting agents, colorants, flavourings, glidants [for example Colloidal Silicon Dioxide (Cab-O—Sil M-5 P)] and disintegrants [for example crospovidone (Polyplasdone XL)].

In one embodiment, the dosage form consists of:

| Ingredients | Unit Formula % w/w |
|---|---|
| Talnetant | 40.0 |
| Sodium Lauryl Sulfate | 0.6 |
| Povidone | 6.0 |
| Erythritol | 15.0 |
| Excipient(s) | to make up 100% |

In one embodiment, the dosage form consists of:

| Ingredients | Unit Formula % w/w |
|---|---|
| Talnetant | 40.0 |
| Sodium Lauryl Sulfate | 0.6 |
| Povidone | 3.4 |
| Erythritol | 20.0 |
| Excipient(s) | to make up 100% |

The effective dose of talnetant depends on the condition of the patient, the frequency and route of administration. A unit dose will generally contain from 20 to 1000 mg of talnetant, in an embodiment 30 to 800 mg, in a further embodiment 200 or 600 mg. The unit dose may be administered one or more times per day (for example 2, 3 or 4 times per day). The total daily dose for a 70 kg adult will normally be in the range 100 to 3000 mg. Alternatively the unit dose will contain from 2 to 20 mg of active ingredient and be administered in multiples, if desired, to give the preceding daily dose.

In an embodiment, the compositions and tablets of the invention are adapted for use in the medical or veterinarial fields. For example, such preparations may be in a pack form accompanied by written or printed instructions for use as an agent in the treatment of the conditions.

NK₃ receptor antagonists, including talnetant, are useful in the treatment and prevention of a wide variety of clinical diseases and conditions characterised by overstimulation of the NK₃ receptors. These diseases and conditions (hereinafter referred to as “diseases and conditions of the invention”) include: CNS disorders such as depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); anxiety disorders (including generalised anxiety disorder, social anxiety disorder, agitation, tension, social or emotional withdrawal in psychotic patients, panic disorder, and obsessive compulsive disorder); phobias (including agoraphobia and social phobia); psychosis and psychotic disorders (including schizophrenia, schizo-affective disorder, schizophreniform diseases, acute psychosis, alcohol psychosis, autism, delerium, mania (including acute mania), manic depressive psychosis, hallucination, endogenous psychosis, organic psychosyndrome, bipolar disorder, paranoid and delusional disorders, puerperal psychosis, and psychosis associated with neurodegenerative diseases such as Alzheimer's diease); post-traumatic stress disorder; attention deficit hyperactive disorder; cognitive impairment (e.g. the treatment of impairment of cognitive functions including attention, orientation, memory (memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states)); convulsive disorders such as epilepsy (which includes simple partial seizures, complex partial seizures, secondary generalised seizures, generalised seizures including absence seizures, myoclonic seizures, clonic seizures, tonic seizures, tonic clonic seizures and atonic seizures); psychosexual dysfunction (including inhibited sexual desire (low libido), inhibited sexual arousal or excitement, orgasm dysfunction, inhibited female orgasm and inhibited male orgasm, hypoactive sexual desire disorder, female sexual desire disorder, and sexual dysfunction side-effects induced by treatment with antidepressants of the SSRI-class); sleep disorders (including disturbances of circadian rhythm, dyssomnia, insomnia, sleep apnea and narcolepsy); disorders of eating behaviours (including anorexia nervosa and bulimia nervosa); neurodegenerative diseases (such as alzheimer's disease, amyotropic lateral sclerosis, motor neuron disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); withdrawal from abuse of drugs including smoking cessation or reduction in level or frequency of such activities (such as abuse of cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine, methylamphetamine or a combination thereof); pain (which includes neuropathic pain (including diabetic neuropathy; sciatica; non-specific lower back pain; multiple sclerosis pain; pain associated with fibromyalgia or cancer; AIDS-related and HIV-related neuropathy; chemotherapy-induced neuropathy; neuralgia, such as post-herpetic neuralgia and trigeminal neuralgia; sympathetically maintained pain and pain resulting from physical trauma, amputation, cancer, toxins or chronic inflammatory conditions such as rheumatoid arthritis and osteoarthritis; reflex sympathetic dystrophy such as shoulder/hand syndrome), acute pain (e.g. musculoskeletal pain, post operative pain and surgical pain), inflammatory pain and chronic pain, pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia), pain associated with migrane, and non-cardiac chest pain); certain CNS-mediated disorders (such as emesis, irritable bowel syndrome and non-ulcer dyspepsia); and pulmonary disorders (such as asthma, chronic obstructive pulmonary disease, airway hyperreactivity and cough).

More diseases or conditions (hereinafter referred to as “preferred diseases and conditions of the invention”) mediated by modulation of the NK₃ receptor include depression; anxiety disorders; phobias; psychosis and psychotic disorders; post-traumatic stress disorder; attention deficit hyperactive disorder; withdrawal from abuse of drugs including smoking cessation or reduction in level or frequency of such activities; irritable bowel syndrome; cognitive impairment; convulsive disorders; psychosexual dysfunction; sleep disorders; disorders of eating behaviours; neurodegenerative diseases; pain; emesis; irritable bowel syndrome; non-ulcer dyspepsia; and pulmonary disorders (such as asthma, chronic obstructive pulmonary disease, airway hyperreactivity and cough).

The following Examples illustrate the present invention.

### Analytical Methods

Particle size distribution of nanomilled suspensions may be directly measured on a Malvern Mastersizer 2000, a Laser Light Diffraction instrument. For the particle size recovery of spray dried powders, a spray dried powder is gently mixed with water (50 mg in 10 mL of water), and the resulting suspension is used for the particle size measurement.

### EXAMPLE 1

Composition 1

| | Composition 1 |
|---|---|
| Ingredients | (unit formula % w/w) |
| Talnetant, micronised | 20.0 |
| Sodium Lauryl Sulfate | 0.300 |
| Povidone (Kollidon 30) | 1.70 |
| Erythritol (Eridex 16952) | 10.0 |
| Purified Water | 68.0 |
| Total | 100.0 |

Sodium lauryl sulphate was dissolved in the water. Talnetant, povidone and erythritol were then added and mixing was continued using a Mixer until a uniform suspension was obtained. The suspension was passed through a Netzsch bead mill until a Dv90 of 450 nm was obtained. The suspension was spray-dried using a Niro Mobile Minor spray dryer (operated in accordance with the manufacturers instructions) at the following settings: two-fluid nozzle: 2 bar atomisation pressure; drying gas flowrate: around 65 m³/hr; suspension spray rate: around 35 to 50 g/min; inlet temperature: around 150° C.; outlet temperature: around 60° C.

The spray dried powder produced from Composition 1 was analysed by X-ray powder diffraction. The instrument was a Philips X'Pert Pro Diffractometer, using the following parameters: scan range 2-40 degrees two-theta; generator power: 40 kV, 40 mA; radiation source: Cu Ka; scan type: continuous; step time: 10.160 seconds; step size: 0.0167 degrees two-theta per step; sample rotation: 25 rpm; incident beam optics: fixed slits at 1 degree with a 0.5 degree removable aperture, 0.04 radian soller slits, 10 mm beam mask; diffracted beam optics: fixed slits (X'celerator module), 0.04 radian soller slits; detector type: Philips X'celerator Real Time Multi Strip.

XRPD analysis confirms that the erythritol in Composition 1 recrystallises upon spray drying and this form matches the starting form.

Furthermore, the particle size recovery of the resulting spray dried powder was nearly complete.

### EXAMPLE 2

Composition 2

| | Composition 2 |
|---|---|
| Ingredients | (unit formula % w/w) |
| Talnetant, micronised | 20.0 |
| Sodium Lauryl Sulfate | 0.300 |
| Povidone (Kollidon 30) | 3.00 |
| Erythritol (Eridex 16952) | 7.50 |
| Purified Water | 69.2 |
| Total | 100.0 |

A spray dried powder from Composition 2 was prepared in a similar manner to Composition 1. XRPD analysis confirms that the erythritol in Composition 2 recrystallises upon spray drying and this form matches the starting form.

The particle size recovery of the resulting spray dried powder was complete.

### EXAMPLE 3

| | Composition 3 |
|---|---|
| Ingredients | (unit formula % w/w) |
| Hydrochlorothiazide | 20.0 |
| Erythritol (Eridex 16952) | 10.0 |
| Povidone (Kollidon 30) | 1.7 |
| Sodium Lauryl Sulfate | 0.3 |
| Purified Water | 68.0 |
| Total | 100.0 |

A spray dried powder from Composition 3 was prepared in a similar manner to Composition 1. XRPD analysis confirms that the erythritol in Composition 3 recrystallises upon spray drying and this form matches the starting form.

### EXAMPLE 4

| | Composition 4 |
|---|---|
| Ingredients | (unit formula % w/w) |
| Naproxen | 20.0 |
| Erythritol (Eridex 16952) | 10.0 |
| Povidone (Kollidon 30) | 1.7 |
| Sodium Lauryl Sulfate | 0.3 |
| Purified Water | 68.0 |
| Total | 100.0 |

A spray dried powder from Composition 4 was prepared in a similar manner to Composition 1. XRPD analysis confirms that the erythritol in Composition 4 recrystallises upon spray drying and this form matches the starting form.

### COMPARATIVE EXAMPLE 1

Composition 5

| | Composition 5 |
|---|---|
| Ingredients | (unit formula % w/w) |
| Talnetant, micronised | 20.0 |
| Sodium Lauryl Sulfate | 0.3 |
| Povidone (Kollidon 30) | 5.0 |
| Lactose (anhydrous) | 10.0 |
| Water | 64.7 |
| Total | 100.0 |

A spray dried powder from Composition 5 was prepared in a similar manner to Composition 1. XRPD analysis of the spray dried powder indicated that the lactose is present in the amorphous form, which is not a thermodynamically stable form.

### COMPARATIVE EXAMPLE 2

| | Composition 6 |
|---|---|
| | (unit formula % |
| Ingredients | w/w) |
| Talnetant, micronised | 20.0 |
| Sodium Lauryl Sulfate | 0.300 |
| Povidone (Kollidon 30) | 1.70 |
| Mannitol Powder (Mannitol 60) | 10.00 |
| Purified Water | 68.0 |
| Total | 100.0 |

A spray dried powder from Composition 6 was prepared in a similar manner to Composition 1. XRPD analysis of the spray dried powder indicated that the mannitol is present as a mixture of the α-mannitol and 8-mannitol forms. These forms differ from the input form (β) which is the thermodynamically stable form at ambient conditions.

1-2. (canceled)

3. A spray-dried pharmaceutical composition comprising (i) a pharmaceutical active, (ii) povidone, (iii) substantially crystalline erythritol and (iv) a surfactant.

4. A pharmaceutical composition comprising (i) talnetant, (ii) povidone, (iii) substantially crystalline erythritol and (iv) a surfactant.

5. A composition as claimed in claim 4, wherein the talnetant is in the form of talnetant particles having a Dv90 in the range from 0.1 to 2.0 μm.

6. A composition as claimed in claim 3, wherein the surfactant is an ionic surfactant or a non-ionic surfactant.

7. A composition as claimed in claim 6 wherein the surfactant is sodium lauryl sulfate or dioctyl sodium sulfosuccinate (docusate sodium).

8. A composition as claimed in claim 3 which also comprises water.

9. A composition as claimed in claim 8 which consists of:

| Ingredients | Unit Formula % w/w |
|---|---|
| Talnetant | 20.0 |
| Sodium Lauryl Sulfate | 0.3 |
| Povidone | 3.0 |
| Erythritol | 7.5 |
| Water | 69.2 |

10. A composition as claimed in claim 8 which consists of:

| Ingredients | Unit formula % w/w |
|---|---|
| Talnetant | 20.0 |
| Sodium Lauryl Sulfate | 0.3 |
| Povidone | 1.7 |
| Erythritol | 10.0 |
| Water | 68.0 |

11. A process for the preparation of a spray-dried composition, the process comprising:
(1) wet milling a dispersion of (i) talnetant, (ii) povidone, (iii) substantially crystalline erythritol and (iv) a surfactant; and then
(2) spray drying or spray granulating the resulting dispersion.

12. A spray dried composition comprising (i) talnetant, (ii) povidone, (iii) substantially crystalline erythritol and (iv) a surfactant.

13. A dosage form comprising a composition as claimed in claim 3.

14. A dosage form as claimed in claim 13 which is for oral administration.

15. A dosage form as claimed in claim 13 which is a capsule or a tablet.

16. A dosage form as claimed in claim 13 which consists of:

| Ingredients | Unit Formula % w/w |
|---|---|
| Talnetant | 40.0 |
| Sodium Lauryl Sulfate | 0.6 |
| Povidone | 6.0 |
| Erythritol | 15.0 |
| Excipient(s) | to make up 100% |

17. A dosage form as claimed in claim 13 which consists of:

| Ingredients | Unit Formula % w/w |
|---|---|
| Talnetant | 40.0 |
| Sodium Lauryl Sulfate | 0.6 |
| Povidone | 3.4 |
| Erythritol | 20.0 |
| Excipient(s) | to make up 100% |

18. A dosage form comprising a composition as claimed in claim 4.

19. A dosage form comprising a composition as claimed in claim 5.

20. A dosage form comprising a composition as claimed in claim 6.

21. A dosage form comprising a composition as claimed in claim 7.

22. A dosage form comprising a composition as claimed in claim 8.

23. A dosage form comprising a composition as claimed in claim 9.

24. A dosage form comprising a composition as claimed in claim 10.

25. A dosage form comprising a composition as claimed in claim 12.

26. A dosage form as claimed in claim 16 which is for oral administration.

27. A dosage form as claimed in claim 17 which is for oral administration.

28. A dosage form as claimed in claim 16 which is a capsule or a tablet.

29. A dosage form as claimed in claim 17 which is a capsule or a tablet.
